(19) United States
(12) Patent Application Publication
(10) Pub. No.: US 2003/0228256 A1
(43) Pub. Date: Dec. 11, 2003

(54) Methods of achieving transplantation tolerance through radioablation of hemolymphopoietic cell populations

(76) Inventors: Luca A. Inverardi, Miami Beach, FL (US); Camillo Ricordi, Miami, FL (US); Giovanni Paganelli, Milano (IT); Aldo N. Serafini, Key Biscayne, FL (US); Jaime Simon, Angleton, TX (US); Alan D. Strickland, Lake Jackson, TX (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/316,790

(22) Filed: Dec. 10, 2002

Related U.S. Application Data
(63) Continuation-in-part of application No. 10/166,053, filed on Jun. 11, 2002.

(51) Int. Cl. A61K 51/00

(52) U.S. Cl. 424/1.49

(57) ABSTRACT

A method for achieving hemolymphopoietic chimerism is disclosed. The method involves the steps of administering to a recipient a bone seeking radiopharmaceutical; transplanting bone marrow-derived cells into the recipient; and transiently suppressing lymphocyte response so as to induce hemolymphopoietic chimerism. The method is useful for decreasing rejection of transplanted organs, tissues or cells and for treating autoimmune diseases. The present invention has the advantage of inducing hemolymphopoietic chimerism without the need for external radiation or harsh cytotoxic drugs. The present invention has the additional advantage of significantly prolonging tolerance to an organ, cell, or tissue transplant.

This application is a continuation-in-part of U.S. application Ser. No. 10/166,053, filed Jun. 11, 2002.

### FIELD OF THE INVENTION

The invention relates to the use of bone agent radiopharmaceuticals, and more particularly those that target bone and can deliver a radiation dose to the bone marrow and bone marrow-derived cells so as to aid in inducing hemolymphopoietic chimerism.

### BACKGROUND OF THE INVENTION

Manipulation of the human immune system has provided several challenges for the medical community, including providing therapies for the treatment of refractory autoimmune diseases, and providing tolerance to organ, tissue and cell transplants. Autoimmune diseases are those wherein a person's immune system mistakenly attacks the cells, tissues and organs of that person's own body. Treatment of refractory autoimmune diseases has been an elusive goal. Bone marrow transplantation is a commonly utilized procedure for the treatment of hematological disorders including malignancies, and has been recently proposed as a therapeutic option for refractory autoimmune diseases. See, for example, Saba N. et al., “Bone marrow transplantation for nonmalignant diseases”,. Journal of Hematother Stem Cell Research 2002 (2):377-87; Furst D., “Stem cell transplantation for autoimmune disease: progress and problems”, Curr Opin Rheumatol. 2002; 14(3):220-4; Oyama Y, Papadopoulos E B, Miranda M, Traynor A E, Burt R K. Allogeneic stem cell transplantation for Evans syndrome. Bone Marrow Transplant. 2001;28(9):903-5; Pratt G, Kinsey S E.Remission of severe, intractable autoimmune haemolytic anaemia following matched unrelated donor transplantation.Bone Marrow Transplant. 2001;28(8):791-3; Berdeja J G, Flinn I W. New approaches to blood and marrow transplantation for patients with low-grade lymphomas. Curr Opin Oncol. 2001;13(5):335-41; Chilton P M, Huang Y, Ildstad S T. Bone marrow cell graft engineering: from bench to bedside. Leuk Lymphoma. 2001;41(1-2):19-34; Burt R K, Slavin S, Burns W H, Marmont A M. Induction of tolerance in autoimmune diseases by hematopoietic stem cell transplantation: getting closer to a cure? Blood. 2002;99(3):768-84.

Transplantation tolerance, defined as complete acceptance of a graft or organ, tissue or cell transplant by an otherwise fully immunocompetent host without the need for long-term immunosuppression, has also been an elusive goal. At present, both chronic and acute graft rejection are alleviated mainly by the use of non-specific immunosuppressive regimens that are often associated with severe complications including development of neoplasms and organ toxicity.

Robust tolerance has been achieved in models that made use of bone marrow or bone marrow-derived cell transplantation. Stable multilineage chimerism achieved following bone marrow or bone marrow-derived cell transplantation often has been considered a prerequisite for donor-specific tolerance induction. Chimerism is defined to mean that two or more tissues of different genetic constitution co-exist together. In the case of hemolymphopoietic chimerism, the blood elements (lymphocytes, platelets, red blood cells and other white cells) of the host and donor co-exist. However, lethal or sub-lethal radiation conditioning strategies commonly used to induce long-term chimerism are often so severely toxic that they preclude the use of these approaches in most clinical conditions other then malignancies or other life-threatening diseases. See, for example, Inverardi L. et al,, “Tolerance and pancreatic islet transplantation”, Philos Trans R Soc Lond B Biol Sci. 2001;356(1409):759-65; Waldmann H., “Therapeutic approaches for transplantation”, Curr Opin Immunol. 2001;13(5):606-10; Sykes M. et al, “Mixed chimerism”, Philos Trans R Soc Lond B Biol Sci. 2001;356(1409):707-26.

Moreover, in order to induce hemolymphopoietic chimerism, many protocols are based on the use of donor bone marrow infusion following the recipient's treatment with potent cytoreductive (lethal or sub-lethal) conditioning protocols, limiting the use of this methodology to the experimental rather then clinical setting, as described in Mayumi H, Good R A., “Induction of tolerance across major barriers using a two-step method with genetic analysis of tolerance induction”, Immunobiology. 1989;179(1):86-108; Ildstad S T, Sachs D H., “Reconstitution with syngeneic plus allogeneic or xenogeneic bone marrow leads to specific acceptance of allografts or xenografts”, Nature. 1984;307(5947):168-70; Sharabi Y, Sachs D H., “Mixed chimerism and permanent specific transplantation tolerance induced by a nonlethal preparative regimen”, J Exp Med. 1989;169(2):493-502; and Colson Y L, Li H, Boggs S S, Patrene K D, Johnson P C, Ildstad S T., “Durable mixed allogeneic chimerism and tolerance by a nonlethal radiation-based cytoreductive approach”, J Immunol. 1996;157(7):2820-9.

Many strategies have been used as recipient preconditioning regimens which include the use of lethal and sub-lethal total body irradiation, thymic and/or lymphoid irradiation, as well as the use of cytotoxic drugs, all aiming at the depletion of the recipient hemolymphopoietic cells in order to “make space” for the engraftment of donor-derived elements as well as to induce transient immunosuppression. As reported in Stewart F M, Crittenden R B, Lowry P A, Pearson-White S, Quesenberry P J, “Long-term engraftment of normal and post-5-fluorouracil murine marrow into normal nonmyeloablated mice”, Blood. 1993;81(10):2566-71 and Rao S S, Peters S O, Crittenden R B, Stewart F M, Ramshaw H S, Quesenberry P J., “Stem cell transplantation in the normal nonmyeloablated host: relationship between cell dose, schedule, and engraftment”, Exp Hematol. 1997;25(2):114-21 (format of citations), the bone marrow has “niches” that support the hemolymphopoietic stem cells via a complex network of cytokines and growth factors, and pre-conditioning might create the necessary “space” for the engraftment of donor-derived hemolymphopoietic stem cells.

However, in the last few years, the concept of “creating space” by the use of whole body irradiation has been challenged. Rather, single or multiple infusions of large doses of donor bone marrow cells in conjunction with co-stimulatory blockade (anti-CD154, B7, CTLA4-Ig), or the use of anti-CD4 and anti-CD8 antibodies along with local thymic irradiation have been proposed. See, for example, Durham M M, Bingaman A W, Adams A B, Ha J, Waitze S Y, Pearson T C, Larsen C P. Cutting edge: administration of anti-CD40 ligand and donor bone marrow leads to hemopoietic chimerism and donor-specific tolerance without cytoreductive conditioning. J Immunol. 2000;165(1):1-4; Pearson T C, Alexander D Z, Hendrix R, Elwood E T, Linsley P S, Winn K J, Larsen C P. CTLA4-Ig plus bone marrow induces long-term allograft survival and donor specific unresponsiveness in the murine model. Evidence for hematopoietic chimerism. Transplantation. 1996;61(7):997-1004; Seung E, Iwakoshi N, Woda B A, Markees T G, Mordes J P, Rossini A A, Greiner D L. Allogeneic hematopoietic chimerism in mice treated with sublethal myeloablation and anti-CD154 antibody: absence of graft-versus-host disease, induction of skin allograft tolerance, and prevention of recurrent autoimmunity in islet-allografted NOD/Lt mice. Blood. 2000;95(6):2175-82; Wekerle T, Kurtz J, Ito H, Ronquillo J V, Dong V, Zhao G, Shaffer J, Sayegh M H, Sykes M. Allogeneic bone marrow transplantation with co-stimulatory blockade induces macrochimerism and tolerance without cytoreductive host treatment. Nat Med. 2000;6(4):464-9; Wekerle T, Sayegh M H, Ito H, Hill J, Chandraker A, Pearson D A, Swenson K G, Zhao G, Sykes M. Anti-CD154 or CTLA4Ig obviates the need for thymic irradiation in a non-myeloablative conditioning regimen for the induction of mixed hematopoietic chimerism and tolerance. Transplantation. 1999;68(9):1348-55; Sharabi Y, Abraham V S, Sykes M, Sachs D H. Mixed allogeneic chimeras prepared by a non-myeloablative regimen: requirement for chimerism to maintain tolerance. Bone Marrow Transplant. 1992;9(3):191-7. (Format of citations) These approaches, although very promising, still rely on either mega doses of donor-bone marrow cells or some form of external irradiation, methods that would be difficult to implement in the clinical setting. U.S. Pat. No. 5,273,738 discloses methods utilizing radioactively labeled antibodies in the targeted irradiation of hemolymphopoietic tissue for use in bone marrow rather than particular subsets of cells. This patent does not recognize the importance of chimerism in inducing tolerance.

U.S. Pat. Nos. 5,514,364; 5,635,156; and 5,876,692 describe the use of cell type-specific antibodies directed to antigens localized on subsets of cells in combination with whole body radiation to enhance chimerism and to increase tolerance induction after donor bone marrow transplantation. These patents do not describe the use of non-immunological radioconjugated compounds, such as phosphonate compounds, for the induction of hemolymphopoietic chimerism.

U.S. Pat. No. 5,902,825 (hereinafter the '825 patent) discloses therapeutic compositions containing an active agent complex formed of a non-radioactive metal ion and an organic phosphonic acid ligand, wherein the metal ion may be a Lanthanide. The '825 patent teaches that such compositions may be used in the treatment of bone diseases and in methods of reducing bone pain, but does not address issues related to bone marrow transplantation. In particular, no suggestion is made to therapeutically target bone marrow or bone marrow-derived cells to achieve chimerism via bone marrow or bone marrow-derived cell transplantation for the induction of tolerance to graft-related antigens. U.S. Pat. No. 5,697,902 (hereinafter the '902 patent) discloses therapeutic compositions and their methods of use in destroying bone-marrow cells in a patient prior to regrafting with normal bone marrow cells. The disclosed method comprises treating a patient with a cytotoxic amount of an antibody or antibody fragment specific to a marker associated with, or produced by, bone marrow cells and which is conjugated to a cytotoxic agent. According to the '902 patent, suitable antibodies are described as being NP-2, MN3, and other antibodies that react with bone marrow cells, such as progenitor cell types. Radioisotopes preferred for therapeutic use with conjugated antibodies include ¹⁵³samarium. This patent discloses a protocol for infusion of autologous bone marrow, but does not address the issues concerning successful induction of transplantation tolerance for achieving hemolymphopoictic chimerism via bone marrow transplantation.

U.S. Pat. No. 6,241,961 (hereinafter the '961 patent) discloses therapeutic radioimmunoconjugates for use in human therapy and methods for their production. According to the '961 patent, radioimmunoconjugates may consist of a monoclonal antibody having binding specificity for CD19, CD20, CD22, HLL2, HLA DR10β, and CD66, conjugated to a radioisotope, and is useful in treating hemolymphopoietic diseases. However, the '961 patent does not suggest the use of non-antibody mediated targeting of bone marrow cells for chimerism induction via bone marrow or bone marrow-derived cell transplantation for tolerance to alloantigens,

U.S. Pat. No. 4,898,724 (hereinafter the '724 patent) teaches the use of Sm-153 with aminophosphonic acid chelators for the treatment of calcific tumors. Administration of chelates such as Sm-153-EDTMP is used to deliver a beta radiation dose to bone tumors. As a result of radioactivity concentration in bone, a dose to bone marrow occurs resulting in a transient bone marrow suppression. However, the '724 patent does not teach or suggest the use of such chelates for chimerism induction.

U.S. Pat. No. 4,882,142 (hereinafter the '142 patent) teaches the use of aminophosphonic acid complexes of radioactive rare earth metal ions such as Sm-153 and Ho-166 for the suppression of bone marrow. A preferred embodiment is the complex formed between the macrocyclic aminophosphonic acid DOTMP and the radioactive metal Ho-166. I.V. injections of these chelates resulted in accumulation of the radioactivity in bone with the effect of suppressing or ablating bone marrow. However the '142 patent does not teach or suggest induction of chimerism.

In WO 0076556 A2, Fritzberg et. al. mention a variety of uses of radioactive bone agents including ablation of the marrow, treating of calcific tumors, and treating autoimmune disease. However, this reference does not teach induction of chimerism. Rather, Fritzberg et. al. propose the use of growth stimulating hormones that would speed up the recovery of bone marrow and as a result would not be conducive to the induction of chimerism..

Therefore, development of suitable protocols that allow the use of low to moderate doses of donor bone marrow or bone marrow-derived cell inoculum, which do not rely on any form of external irradiation or depletion of the peripheral immune system, is necessary to make the induction of tolerance in bone marrow or bone marrow-derived cell recipients clinically practical, without invoking harsh preconditioning regimens and without resulting in unnecessary side effects.

### SUMMARY OF THE INVENTION

In one aspect, the present invention is a method of achieving hemolymphopoietic chimerism comprising administering to a recipient a bone seeking radiopharmaceutical; transplanting bone marrow or bone marrow-derived cells into the recipient; and transiently suppressing lymphocyte response so as to induce hemolymphopoietic chimerism.

In a second aspect, the present invention is a method for decreasing rejection of transplanted organs, tissues or cells comprising administering to a recipient a bone seeking radiopharmaceutical; transplanting bone marrow or bone marrow-derived cells into the recipient; transiently suppressing lymphocyte response; and transplanting one or more organs, tissues or cells.

In a third aspect, the present invention is a method to treat autoimmune disease comprising administering to a recipient a bone seeking radiopharmaceutical; transplanting bone marrow or bone marrow-derived cells into the recipient; and transiently suppressing lymphocyte response.

The present invention has the advantage of inducing hemolymphopoietic chimerism without the need for lethal or sub-lethal conditioning regimens as used in some of the methods described in the above identified prior art. The use of bone-seeking radioactive compounds represents a viable approach to creating the “space” required for the donor stem cellengraftment and hemolymphopoietic chimerism without the need for external radiation or harsh cytotoxic drugs. The method of the present invention also provides more certainty that hemolymphopoietic chimerism will indeed result, as opposed to some of the methods of the prior art that do not provide an environment allowing induction of chimerism to an adequate degree. And, using the method of the present invention, tolerance to an organ, cell, or tissue transplant can be achieved.

### BRIEF DESCRIPTION OF THE DRAWINGS

The invention is further illustrated by the following Figures, wherein:

FIG. 1 graphically depicts the results of treating mice with a single dose, IV, of ¹⁵³Sm-EDTMP, 150 μCi or 500 μCi, prior to administration of 20×10⁶ or 100×10⁶ allogeneic donor bone marrow-derived cells (BMC) as a single intravenous (IV) dose;

FIG. 2 graphically shows that a single administration of BMC resulted in bone marrow engraftment in all recipients analyzed;

FIG. 3 graphically shows the percentage of donor-derived cells in recipients treated with 20×10⁶ BMC, anti-CD154 mAb, and one of 4 conditioning approaches;

FIG. 4 shows the percentage of donor-derived cells in control animals treated with 100×10⁶ BMC and one of the 4 conditioning approaches;

FIG. 5 shows the percentage of donor-derived cells in the control animals treated with 20×10⁶ BMC, and one of the 4 conditioning approaches;

FIG. 6 shows the percent of donor-derived cells in the control animals treated with 20×10⁶ BMC or 100×10⁶ BMC along with anti-CD154 mAb (in the absence of ¹⁵³Sm-EDTMP treatment);

FIG. 7 depicts a two-color flow cytometric analysis of the proportion of donor-derived lymphoid (B cells), NK, and myeloid (granulocytes) lineages in representative mixed chimeras prepared using a non-lethal conditioning regiment of 20×10⁶ BMC, ¹⁵³Sm-EDTMP, and anti-CD154 mAb (upper panels) as well as 20×10⁶ BMC and anti-CD154 mAb (lower panels);

FIG. 8 depicts a two-color flow cytometric analysis of the proportion of donor-derived lymphoid (B cells), NK, and myeloid (granulocytes) lineages in representative mixed chimeras prepared using a non-lethal conditioning regiment of 100×10⁶ BMC, ¹⁵³Sm-EDTMP, and anti-CD154 mAb (upper panels) as well as 100×10⁶ BMC and anti-CD154 mAb (lower panels);

FIG. 9 graphically shows the survival of full thickness tail-derived skin grafts placed on the recipients treated with 20×10⁶ BMC, ¹⁵³Sm-EDTMP, and anti-CD154 mAb, or the indicated control groups; and

FIG. 10 graphically depict the survival of full thickness tail-derived skin grafts placed on the recipients treated with 100×10⁶ BMC, ¹⁵³Sm-EDTMP, and anti-CD154 mAb, or the indicated control groups.

### DETAILED DESCRIPTION OF THE PREFERRED EMBODIMENTS

The present invention focuses on a novel approach of attaining a profound, but transient myelodepression by selectively targeting the recipient bone marrow in order to achieve multilineage chimerism. In one embodiment, the present invention can be used to obtain multilineage hemolymphopoietic chimerism. The term “multilineage” is defined herein to mean that more than one cell lineage derived from bone marrow-contained precursors is detectable in the recipient. The present invention has particular applicability to inducing transplantation tolerance in a recipient of an organ, tissue or cell transplant, and to treating autoimmune diseases.

In one embodiment, hemolymphopoietic chimerism is induced so as to provide immunological tolerance to at least one member of the group consisting of alloantigens, autoantigens and xenoantigens. Alloantigens are those antigens recognized by the immune system that are expressed on cells, tissues or organs of a non-identical individual of the same species. Autoantigens are those antigens expressed by an individual's tissues, cells or organs that elicit an autoimmune response or that are the target of an autoimmune disease. Xenoantigens are those antigens recognized by the immune system that are expressed on cells, tissues or organs of an individual of a different species.

The method of the present invention includes the step of administering a bone seeking radiopharmaceutical to a recipient. A bone seeking radiopharmaceutical is defined herein to mean a complex of a radionuclide and a ligand which targets bone rather than soft tissue. Preferably, the radiopharmaceutical comprises a rare earth radionuclide complexed with an aminophosphonic acid. Preferred radionuclides include Sm-153Ho-166, Gd-159, Lu-177, Dy-165, Y-90, In-155m, Re-186, Re-188, Sn-117m, La-140,I-131, Cu-67, Ac-225, Bi-212, Bi-213, At-211, Ra-223, Pm-149, Rh-105; Au-198, Au-199, Dy-166, Sc-47, Yb-175, P-32, Sr-89, Ir-192, Tb-149, and Ra-224.

Preferred ligands include aminophosphonic acids and lower carboxylic acids. More preferably, the ligand is selected from the group consisting of ethylenediaminetetramethylenephosphonic acid (EDTMP), diethylenetriaminepentamethylenephosphonic acid (DTPMP), hydroxyethylethylenediaminetrimethylenephosphonic acid (HEEDTMP), nitrilotrimethylenephosphonic acid (NTMP), tris(2-aminoethyl)aminehexamethylenephosphonic acid (TTHMP), 1-carboxyethylenediaminetetramethylenephosphonic acid (CEDTMP) and bis(aminoethylpiperazine)tetramethylenephosphonic acid (AEPTMP),. Ethylenediaminetetraacetic acid (EDTA), 1,4,7,10-tetraazacyclododecane-N,N′,N″,N′″-tetramethylenephosphonic acid (DOTMP), hydroxyethyldiphosphonic acid (HEDP), methylenediphosphonic acid (MDP), diethylenetriaminepentaacetic acid (DTPA), hydroxethylethylenediaminetriacetic acid (HEDTA), and nitrilotriacetic acid (NTA). Preferably, the bone seeking radiopharmaceutical complex is chosen from the group consisting of Sm-153-EDTMP, Sm-153-DOTMP, Ho-166-EDTMP, Ho-166-DOTMP, Gd-159-EDTMP, Gd-159-DOTMP, Dy-165-EDTMP, Dy-165-DOTMP, Re-186-HEDP, Re-188-HEDP, and Sn-117m-DTPA.

Certain bone seeking radiopharmaceuticals which can be used in the method of the present invention do not require the use of a chelating agent. For example P-32 can be used alone as a bone seeking radiopharmaceutical without a ligand. Also, Sr-89 as the chloride can be used, as indicated in Robinson R G, Spicer J A, Preston D F, et al., “Treatment of Metastatic Bone Pain With Strontium-89,” Nucl. Med. Biol. 14:219-222 (1987). Most preferred radiopharmaceuticals for use with the present invention include Sm-153-EDTMP, Sm-153-DOTMP, Ho-166-EDTMP, Ho-166-DOTMP, Gd-159-EDTMP, and Gd-159-DOTMP. Examples of these complexes are described in U.S. Pat. Nos. 4,976,950, 4,882,142, 5,059,412, 5,066,478, 5,064,633, 4,897,254, 4,898,724, and 5,300,279, which are incorporated herein by reference.

The bone seeking radiopharmaceutical may be introduced to a human bone marrow recipient in dosages ranging from about 1 mCi/Kg to about 50 mCi/Kg. The dose of the bone seeking radiopharmaceutical will depend upon the nuclear properties of the radionuclide, the localization of the radiopharmaceutical in bone, and the localization in other tissues. For example, an isotope with a long half life and a high energy emission would deliver a higher dose than one with a short half life and low energy emissions. In addition, a lower, diagnostic dose of the radiopharmaceutical may be used to determine the biodistribution of the bone seeking radiopharmaceutical allowing for an estimation of the dose prior to administration of the higher doses. For Sm-153-EDTMP, a dose of from about 3 miCi/Kg (111 MBq/Kg) to about 20 mCi/Kg (740 MBQ) is preferred. More preferred is a dose of about 6 mCi/Kg (222 MBq/Kg) to about 10 mCi/Kg (370 MBq/Kg) body weight. Each radionuclide and the form that it is administered will give a different dose. The dose is dependent on the decay properties of the radionuclide and the biodistribution. Preferred doses to the red bone marrow are from about 800 rads (8 Grey) to about 5,000 rads (50 Grey). More preferred is from about 1600 Rads (16 Grey) to about 3,000 rads (30 Grey).

A single administration of the radioactive complexes should be satisfactory for inducing chimerism following bone marrow or bone marrow-derived cell transplantation, although multiple dose regimens may be employed, when necessary. Radioactivity will remain in recipient bone, thereby affecting the bone marrow or bone marrow-derived cells therein, for the life of the isotope. Thus, while Sm-153, Ho-166, and Gd-159 are preferred, other radioactive isotopes having relatively short, but clinically appropriate, half-lives may also be employed in complexes useful for the invention. Suitable complexes may be prepared according to known protocols optionally utilizing complex forming agents, or may be obtained from commercial sources.

The radiopharmaceuticals may be formulated into any pharmaceutically acceptable dosage form, including liquids, emulsions, suspensions and the like. Liquid solutions for injection are particularly preferred. Pharmaceutical compositions of the complexes for use according to the invention may also contain suitable diluents, excipients, buffers, stabilizers and carriers. Sterile water or sterile isotonic saline solutions are particularly preferred. Another step in the method of the present invention entails transplanting the bone marrow-derived cells into a recipient. The term “bone marrow-derived cells” is defined herein to mean bone marrow cells, stem cells and precursors as well as cells obtained from the bone marrow and selected or manipulated in vitro (e.g. cultured, enriched etc) as well as cells with stem cell/precursor cell properties obtained from other anatomical sources (peripheral blood after mobilization, cord blood etc). Bone marrow-derived cells are transplanted into the recipient via protocols known to those of skill in the art.

The method of the present invention further comprises the step of transiently suppressing the lymphocyte response. Transient lymphocyte response suppression is defined to mean that the treatment is transient, or of a relatively short duration, as opposed to being chronic in duration. Preferably, chronic lymphocyte response suppression is avoided with the use of the present invention, so as to minimize the side effects associated with such chronic lymphocyte suppression.

For the step of transiently suppressing lymphocyte response, a biological modifier is administered to the host. Suitable biological modifiers include antibodies, cytokines, immunosuppressive drugs, peptides, proteins, nucleic acids or a combination thereof. Most preferably, the bone marrow-derived cells are transplanted in conjunction with at least one antibody raised against an antigen selected from the group consisting of CD4, CD8, CD3, CD5, CD55, CD40, CD40L, B7.1, B7.2, CD28, and LFA-1.

Appropriate dosage levels and length of administration of the biological modifier can be determined by those of ordinary skill in the art and will depend upon factors such as histocompatability matching, dose of transplanted cells, age of the patient, and so forth. In one embodiment, the method of the present invention is useful for decreasing rejection of transplanted organs, tissues or cells. The organs, tissues or cells can be transplanted using procedures known to those skilled in the art. Organs, tissues or cells for which transplantation tolerance can be enhanced by the present invention include liver, heart, lung, kidney, intestine, pancreas, larynx, blood vessels limbs, endocrine organs, skin, islet cells, cornea, nerves, muscles, keratinocytes and keratynocyte precursors, chondrocytes and condrocyte precursors hepatocytes and hepatocyte precursors, myocytes and myoblasts including cardiomyocytes and cardiomyoblasts, neural cells and neural cell precursors, endothelial cells, endocrine cells and endocrine cell precursors, stem cells and cells of different lineage derived from stem cells. In another embodiment, the method of the present invention can be used to treat autoimmune disease. In this embodiment, the bone marrow-derived cells that are transplanted during the bone marrow-derived cell transplantation step can be autologous or homologous. Also, the bone marrow-derived cells can be either unmanipulated or depleted of mature T-lymphocytes prior to transplantation.

Autoimmune diseases typically affect the nervous system, cardiac system, the eye, cardiac system, respiratory system, urogenital system, gastrointestinal system, blood, blood vessels, endocrine glands, skin, and musculoskeletal system, including connective tissue diseases. The autoimmune diseases that can be treated using the method of the present invention include rheumatoid arthritis, ankylosing spondilytis polymyositis and dermatomyositis systemic lupus erythematosus, vasculitides, Goodpasture's syndrome Wegener granulomatosis uveitis Sjogren's syndrome Bechet's disease, autoimmune myocarditis and perycarditis, multiple sclerosis, inflammatory bowel disease, Crohn's disease, ulcerative colitis, autoimmune gastritis, autoimmune hepatitis primary biliary chirrosis, diabetes, autoimmune thyroid disease Graves disease, Hashimoto thyroiditis, Addison's disease, ipoparathyroidism, autoimmune hypophysitis, ovaritis, myastenia gravis, alopecia areata universalis, vitiligo, psoriasispemphigus p, p scleroderma, and autoimmune diseases of the blood such as Henoch-Schonlein purpura, autoimmune hemolytic anemia etc and other disease due to the presence of immune complexes where the antigen is an autoantigen.

An advantage of the protocols according to the invention over conventional therapies for bone marrow reduction prior to transplantation is the elimination of cumbersome steps required for conjugating radioisotopes to antibodies. Thus, tolerance induction or immunosuppression according to certain preferred embodiments of the invention can be successfully implemented in an efficient manner not previously recognized in the art. In vivo testing of the inventive method using a bone seeking radiopharmaceutical to target bone produced surprising success in inducing myelosuppression in a highly selective manner to achieve chimerism upon bone marrow-derived allotransplantation, as described in the Figures and Example herein below:

The invention is further described in the following non-limiting Example.

### EXAMPLE

Methods

Animals. All animal procedures were performed under the supervision and approval of the University of Miami Institutional Animal Care and Use Committee (IACUC). Mice (7-8 week old Balb/c (H-2^{d}), C57BL/6 (B6; H-2^{b}) and C3H/HeJ (C3H; H-2^{k})) were purchased from Jackson Laboratories (Bar Harbor, Me.). Recipient C57BL/6 mice were used at 9-10 weeks of age. All animals were housed in pathogen-free room in sterile microisolator cages with autoclaved feed and autoclaved acidified water.

Bone marrow-derived Cell Transplantation. Balb/c mice, 8-9 weeks old, used as donors, were sacrificed on the day of the transplant. Bone marrow cells (BMC) were prepared according to a previously published regimen. Briefly, after removing femura and tibiae, and cleaning them from muscle tissue and cartilage, BMC were flushed with sterile RPMI-1640 (Mediatech, Inc, Herndon, Va.) supplemented with 0.8 mg/ml Gentamycin (Gibco, Gaithersburg, Md.), using 23G needle. BMC were filtered through a sterile nylon mesh and counted. Fully MCH-mismatched C57BL/6 recipients, 9-10 weeks of age, were injected intravenously with either 20×10⁶ or 100×10⁶ unmanipulated BMCs resuspended in 0.5 and 1.0 ml of HBSS Hank's Balanced Saline Solution) (Mediatech) respectively, on either day 7 or 14. Tolerance induction protocol consisted of either 150 or 500 μCi of ¹⁵³Sm-EDTMP (Berlex Laboratories Wayne, N.J.), I.V., on day—7, and 0.5 mg hamster anti-murine CD154 mAb (MR-1), purchased from Taconic (Germantown, N.Y.) administered intraperitoneally (I.P.) on days—1,0,7,14, 21 and 28.

Skin grafting. Full-thickness skin donor (Balb/c) and third party (C3H/HeJ) grafts were transplanted onto the lateral thoracic area of the recipients either the day following BMC-Tx, or 4 weeks following the last administration of MR-1 mAb, using techniques described previously. Briefly, square, full-thickness skin grafts (1 cm²) were prepared from the tail skin of donors. Graft beds were prepared on the right (donor-specific) and left (third party) lateral thoracic wall of recipient mice. Grafts were fixed to the beds with 4 sutures of 5.0 silk at the corners of the graft and covered with a petroleum jelly-coated gauze and a plaster cast. The grafts were first inspected on the eighth-day following grafting, and every third day thereafter. Graft rejection was considered complete when no viable graft tissue was detected by visual inspection. Recipient mice were considered to be tolerant when donor-specific skin grafts survived in perfect condition for <150 days.

Immunohemotyping of chimeras. Engraftment of donor-derived BMCs was ascertained by flow cytometric analysis (FCM) of recipient peripheral blood mononuclear cells (PBMCs), splenocytes, thymocytes and bone marrow cells after staining with FITC-conjugated anti-mouse H-2K^{b} or H-2K^{d} and Cy-Chrome-conjugated CD3 monoclonal antibodies (mAbs) purchased from PharMingen (San Diego, Calif.) at multiple time points during the experiment as well as at sacrifice. Cells were also assessed for non-specific staining using an Ig isotype control (FITC-conjugated mouse IgG₂ₐ and Cy-Chrome-conjugated rat IgG_{2b}), and the percentage of cells stained with this Ab was subtracted from the values obtained from staining with the specific Ab to determine the relative number of positive cells. Reconstitution of various cell lineages was assessed using FITC-conjugated anti-mouse H-2K^{b} or H-2K^{d} and PE-conjugated anti-mouse CD19/CD22 in the B cell, PE-conjugated anti-mouse Ly-6G in the granulocyte, and PE-conjugated anti-mouse Mac-3 in the macrophage compartments. Recipient animals were first tested 1 week after BMC-Tx, every 2 weeks up to 6 weeks, and every 4 weeks thereafter. Purified anti-mouse CD16/CD32 (FcγIII/II) was used to block non-specific binding to the Fc receptors. FCM analyses were preformed using CellQuest software on a FACScan cytometer purchased from Becton Dickinson & Co. (Mountain View, Calif.).

Analysis of various T cell receptor families. Splenocytes were used to analyze the expression of Vb3⁺, Vb5⁺, Vb11⁺ and Vb14⁺ families in the chimeras at the time of sacrifice. For two-color analysis, cells were blocked with purified anti-mouse CD16/CD32 (FcγIII/II) (PharMingen), and then incubated with FITC-conjugated H-2K^{d} and PE-conjugated anti-Vb3⁺, Vb5⁺, Vb11⁺ or Vb14⁺ (PharMingen) for 30 minutes on ice. FITC-conjugated mouse IgG2a, PE-conjugated Armenian Hamster IgG, group 2, mouse IgG1, rat IgG2b and rat IgM antibodies (PharMingen) were used as negative controls.

Mixed Lymphocyte Reaction. Splenocytes depleted of red blood cells were incubated at 37° C. in 5% CO₂ for 3 days in quintuplicate wells containing 2×10⁵ responders with 2×10⁵ stimulators treated with Mytomicin C (Sigma, St. Louis, Mo.) in Iscove's tissue culture media (Gibco, Gaithersburgh, Md.) containing 10% heat-inactivated FCS, 2 mM L-Glutamine (Mediatech), 25 mM HEPES (Mediatech) and 0.05 mM β-mercaptoethanol. Responder cells from chimeric mice and stimulator splenocytes, BMCs and keratinocytes were incubated for 3 days in a 96 round-bottom tissue culture plates, and then pulsed with 1 μCi [³H] thymidine; [³H] thymidine incorporation was assessed after 8 hours. Stimulation indices were calculated by dividing mean counts per minute (c.p.m.) by responses against self.

Staining for the presence of anti-donor antibodies. 1×10⁶ splenocytes, isolated from naïve Balb/c donors were incubated with several different dilutions (1:3; 1:10; 1:30; 1:100) of plasma from the chimeric recipients at 4° C. for 60 minutes. Cells were washed with PBS supplemented with 1% BSA, 0.02% sodium azide, and then incubated with FITC-conjugated goat anti-mouse IgG (H+L) (Jackson ImmunoResearch Laboratories, West Grove, Pa.) and PE-conjugated anti-mouse CD22 for 30 minutes on ice. The cells were then washed with PBS and analyzed on a Becton Dickinson FACScan. Plasma from a naïve C57BL/6 incubated with splenocytes from naïve Balb/c donors was used as a baseline.

Results

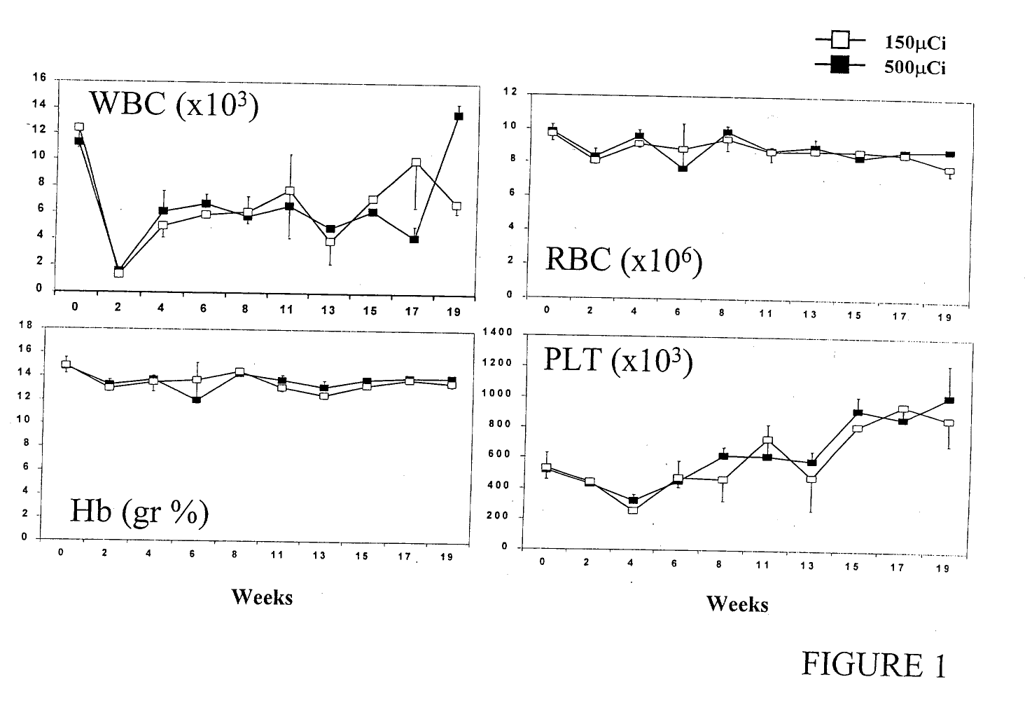

Recipient animals (C57BL/6, H-2^{b}) were treated with a single IV dose of ¹⁵³Sm-EDTMP, 150 μCi or 500 μCi, prior to administration of 20×10⁶ or 100×10⁶ allogenic donor bone marrow cells (BMC) (BALB/c, H-2^{d}), also administered as a single IV dose. BMC transplantation (BMC-Tx) was performed on day 7 or 14 following the administration of ¹⁵³Sm in the presence of transient T lymphocyte co-stimulatory blockade by MR-1 (hamster anti-murine CD154 mAb) on days—1, 0, 7, 14, 21 and 28, 0.5 mg IP. The lower dose of ¹⁵³Sm, 150 μCi, proved to be as effective as the higher dose, 500 μCi. Treatment with ¹⁵³Sm-EDTMP resulted in transient myelodepression that occurred one week post administration of the compound and was spontaneously resolved by 4-6 weeks post-administration, as shown in FIG. 1. Both the 150 μCi and 500 μCi doses of ¹⁵³Sm-EDTMP have similar effect on hemolymphopoietic elements. Although there is a marked myelodepression, as assessed by a decreased white blood cell counts (WBC), administration of ¹⁵³Sm-EDTMP does not have significant effect on red blood cell (RBC), hemoglobin (Hb), and Platelet (PLT) counts. Similar data were obtained in animals treated with ¹⁵³Sm-EDTMP and not transplanted with allogeneic BMC (not shown). Thus, ¹⁵³Sm-EDMP leads to a transient myelodepression of the WBC compartment, which is spontaneously reversible either in the presence or absence of an allogeneic BMC-Tx. No dramatic alterations of RBC, PLT or Hb counts were evident.

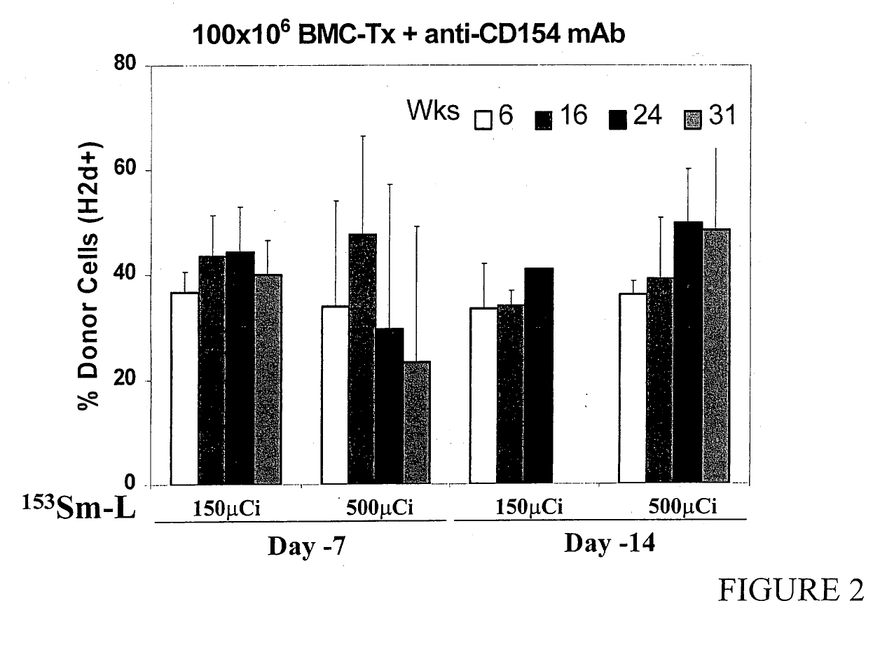

Single administration of BMC resulted in BM engraftment in all recipient animals analyzed. FIG. 2 shows percentages of donor-derived cells in the recipients treated with 100×10⁶ BMC, anti-CD154 mAb, and one of 4 conditioning approaches—¹⁵³Sm-EDTMP 150 μCi, followed by administration of BMC on day 7; ¹⁵³Sm-EDTMP 500 μCi, followed by administration of BMC on day 7, ¹⁵³Sm-EDTMP 150 μCi, followed by administration of BMC on day 14; and ¹⁵³Sm-EDTMP 500 μCi, followed by administration of BMC on day 14. Typing of PBL obtained from the recipient animals starting at 2 weeks following the reconstitution with donor-derived BM allogeneic cells, every two weeks up to 6 weeks post-reconstitution, and every 4 weeks afterwards was performed using anti Class I H-2^{b}-FITC and H-2^{d}-FITC. Analysis was performed on the lymphoid gate, and the values were normalized to 100%. CD3+ T lymphocytes of donor origin were also present, suggesting mixed chimerism of the lymphoid lineage as well.

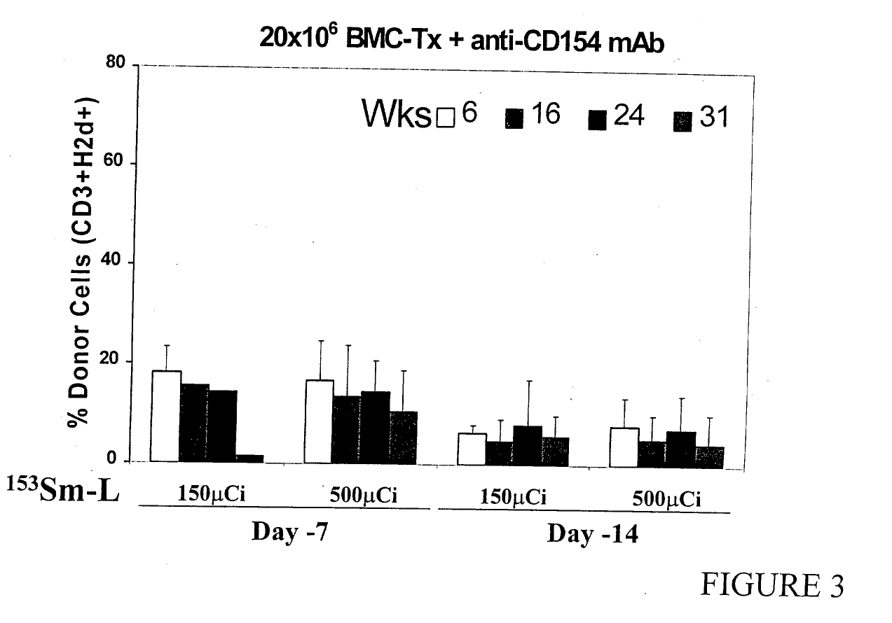

In FIG. 3 is shown the percentage of donor-derived cells in the recipients treated with 20×10⁶ BMC, anti-CD154 mAb, and one of the 4 conditioning approaches: ¹⁵³Sm-EDTMP 150 μCi, followed by administration of BMC on day 7; ¹⁵³Sm-EDTMP 500 μCi, followed by administration of BMC on day 7; ¹⁵³Sm-EDTMP 150 μCi, followed by administration of BMC on day 14; and ¹⁵³Sm-EDTMP 500 μCi, followed by administration of BMC on day 14. Typing of PBL obtained from the recipient animals starting at 2 weeks following the reconstitution with donor-derived BM allogeneic cells, every two weeks up to 6 weeks post-reconstitution, and every 4 weeks afterwards was performed using anti Class I H-2^{b}-FITC and H-2^{d}-FITC. Analysis was performed on the lymphoid gate, and the values were normalized to 100%. CD3+ T lymphocytes of donor origin were also present, suggesting mixed chimerism of the lymphoid lineage as well.

Therefore, administration of ¹⁵³Sm-EDMP in the presence of costimulatory blockade leads to long-lasting hemolymphopoietic chimerism in the recipients of allogeneic BMC. The dose of ¹⁵³Sm-EDMP (150 μCi vs. 500 μCi) and the timing of BMC-Tx relative to ¹⁵³Sm-EDMP administration do not grossly influence the results. BMC dose, on the other hand, directly correlates with the levels of chimerism achieved.

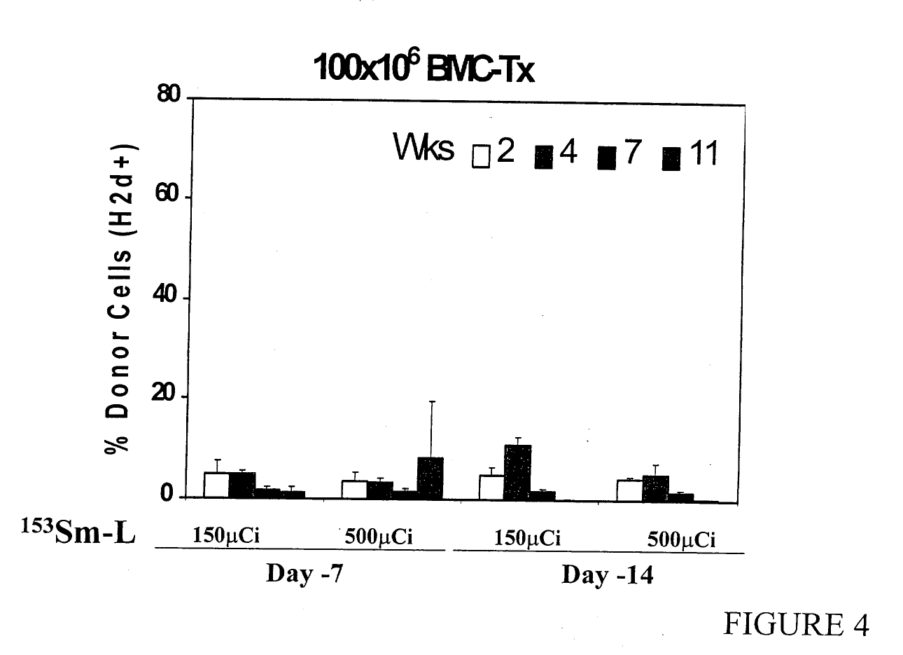

As shown in FIG. 4, the percentage of donor-derived cells in the control animals treated with 100×10⁶ BMC and one of the 4 conditioning approaches was assessed. The conditioning regimens were ¹⁵³Sm-EDTMP 150 μCi, followed by administration of BMC on day 7; ¹⁵³Sm-EDTMP 500 μCi, followed by administration of BMC on day 7; ¹⁵³Sm-EDTMP 150 μCi, followed by administration of BMC on day 14; and ¹⁵³Sm-EDTMP 500 μCi, followed by administration of BMC on day 14. This fourth regimen differs from the previous, since no anti-CD154 mAb to induce costimulatory blockade was used. Typing of PBL obtained from the recipient animals starting at 2 weeks following the reconstitution with donor-derived BMC allogeneic cells, every two weeks up to 6 weeks post-reconstitution, and every 4 weeks afterwards was performed using anti Class I H-2^{b}-FITC and H-2^{d}-FITC. Analysis was performed on the lymphoid gate, and the values were normalized to 100%.

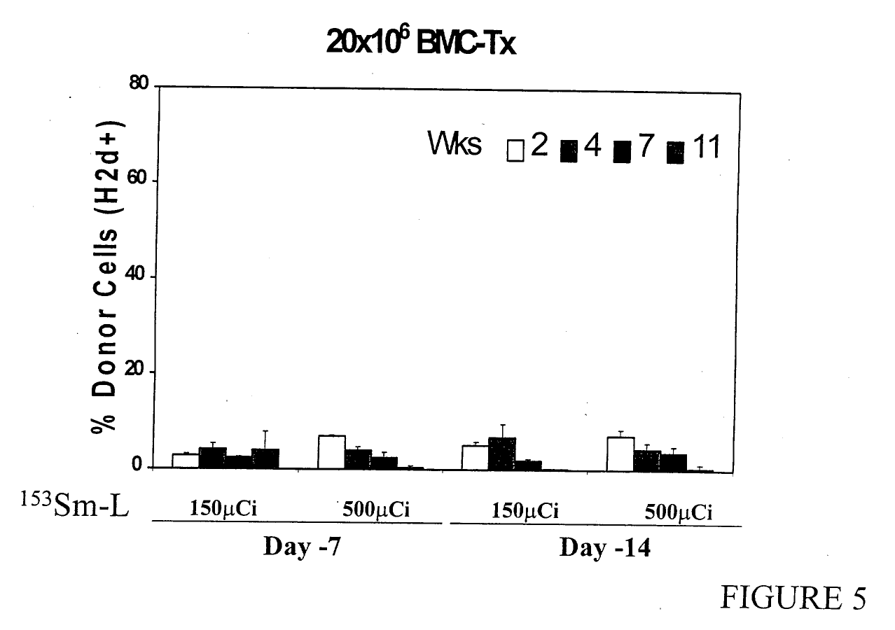

FIG. 5 shows the percent of donor-derived cells in the control animals treated with 20×10⁶ BMC, and one of the 4 conditioning approaches: ¹⁵³Sm-EDTMP 150 μCi, followed by administration of BMC on day 7; ¹⁵³Sm-EDTMP 500 μCi, followed by administration of BMC on day 7; ¹⁵³Sm-EDTMP 150 μCi, followed by administration of BMC on day 14; and ¹⁵³Sm-EDTMP 500 μCi, followed by administration of BMC on day 14 (this regimen differs from the previous, since no anti-CD154 mAb to induce costimulatory blockade was used). Typing of PBL obtained from the recipient animals starting at 2 weeks following the reconstitution with donor-derived BMC allogeneic cells, every two weeks up to 6 weeks post-reconstitution, and every 4 weeks following that was performed using anti Class I H-2^{b}-FITC and H-2^{d}-FITC. Analysis was performed on the lymphoid gate, and the values were normalized to 100%.

Thus, the data from FIGS. 4-5 show that in the absence of co-stimulatory blockade, ¹⁵³Sm-EDMP administration followed by BMC-Tx only leads to transient chimerism, regardless of the dose of BMC (20×10⁶ or 100×10⁶).

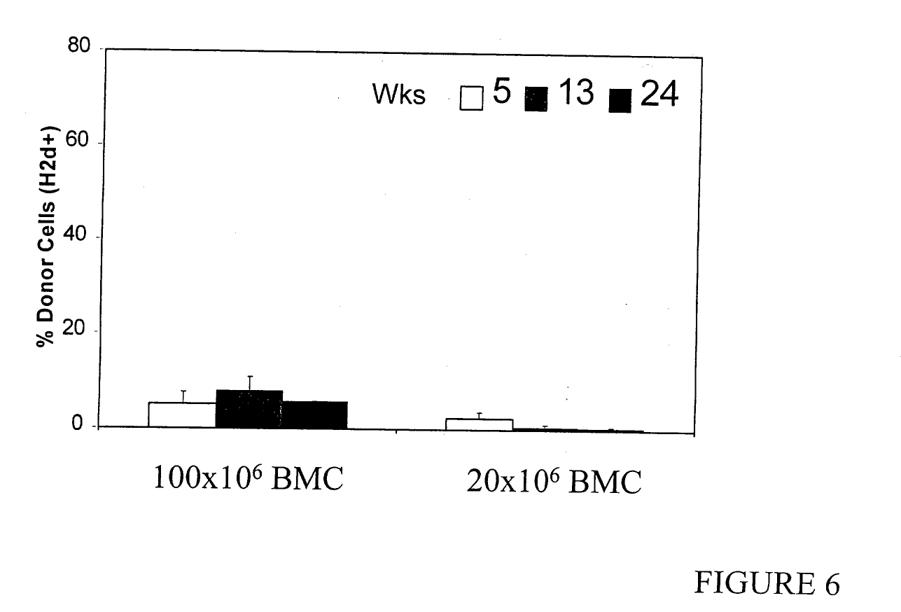

The percentage of donor-derived cells in the control animals treated with 20×10⁶ BMC or 100×10⁶ BMC along with anti-CD154 mAb (in the absence of ¹⁵³Sm-EDTMP treatment) is shown in FIG. 6. Typing of PBL obtained from the recipient animals starting at 2 weeks following the reconstitution with donor-derived BMC allogeneic cells, every two weeks up to 6 weeks post-reconstitution, and every 4 weeks following that was performed using anti Class I H-2^{b}-FITC and H-2^{d}-FITC. Analysis was performed on the lymphoid gate, and the values were normalized to 100%. The results indicate that treatment with BMC-Tx and co-stimulatory blockade without administration of ¹⁵³Sm-EDMP, leads to transient chimerism when a low dose (20×10⁶) BMC is administered and to low level, stable chimerism when 100×10⁶ BMC are administered.

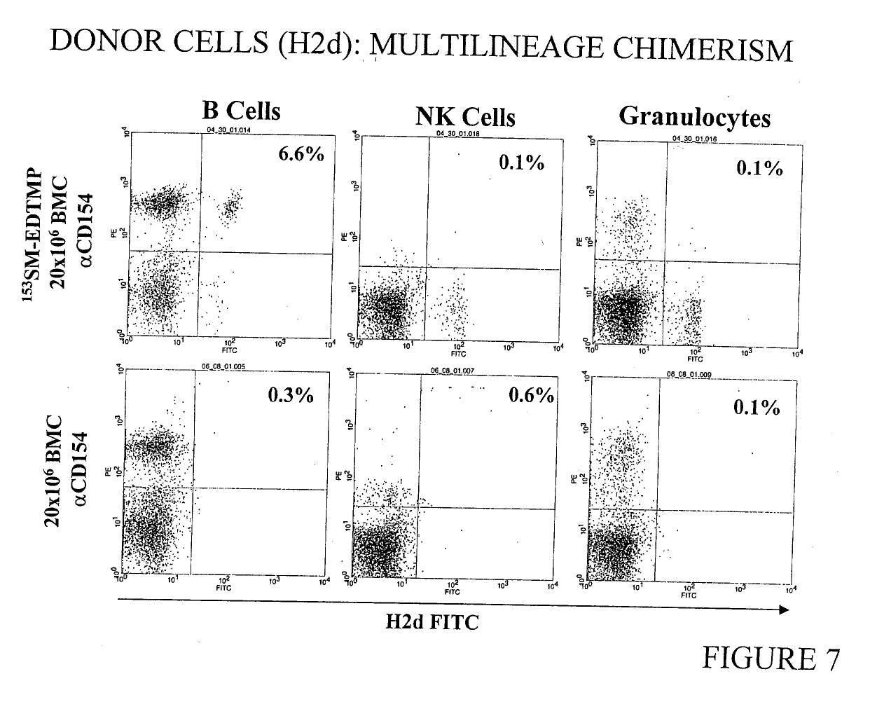

FIG. 7 shows a two-color flow cytometric analysis of the proportion of donor-derived lymphoid (B cells), NK, and myeloid (granulocytes) lineages in representative mixed chimeras prepared using a non-lethal conditioning regiment of 20×10⁶ BMC, ¹⁵³Sm-EDTMP, and anti-CD154 mAb (upper panels) as well as 20×10⁶ BMC and anti-CD154 mAb (lower panels). Analysis was performed using Class I H-2^{d}-FITC and either CD22 (B cells), NK, or GRAN1 (granulocytes), all PE. Analysis was performed on the lymphoid gate, and the values were normalized to 100%.

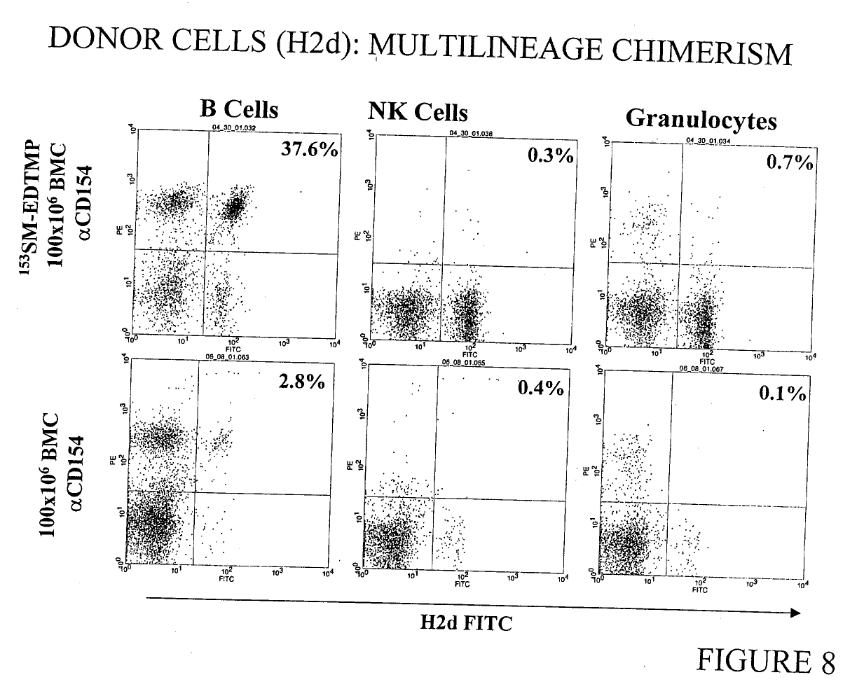

In FIG. 8 is shown a two-color flow cytometric analysis of the proportion of donor-derived lymphoid (B cells), NK, and myeloid (granulocytes) lineages in representative mixed chimeras prepared using a non-lethal conditioning regiment of 100×10⁶ BMC, ¹⁵³Sm-EDTMP, and anti-CD154 mAb (upper panels) as well as 100×10⁶ BMC and anti-CD154 mAb (lower panels). Analysis was preformed using Class I H-2^{d}-FITC and either CD22 (B cells), NK, or GRAN1 (granulocytes), all PE. Analysis was performed on the lymphoid gate, and the values were normalized to 100%.

As is evident from the data presented in FIGS. 7 and 8, long-term, stable multilineage chimerism is achieved in the group treated with a combination of BMC-Tx, ¹⁵³Sm-EDMP, and anti-CD154 mAb.

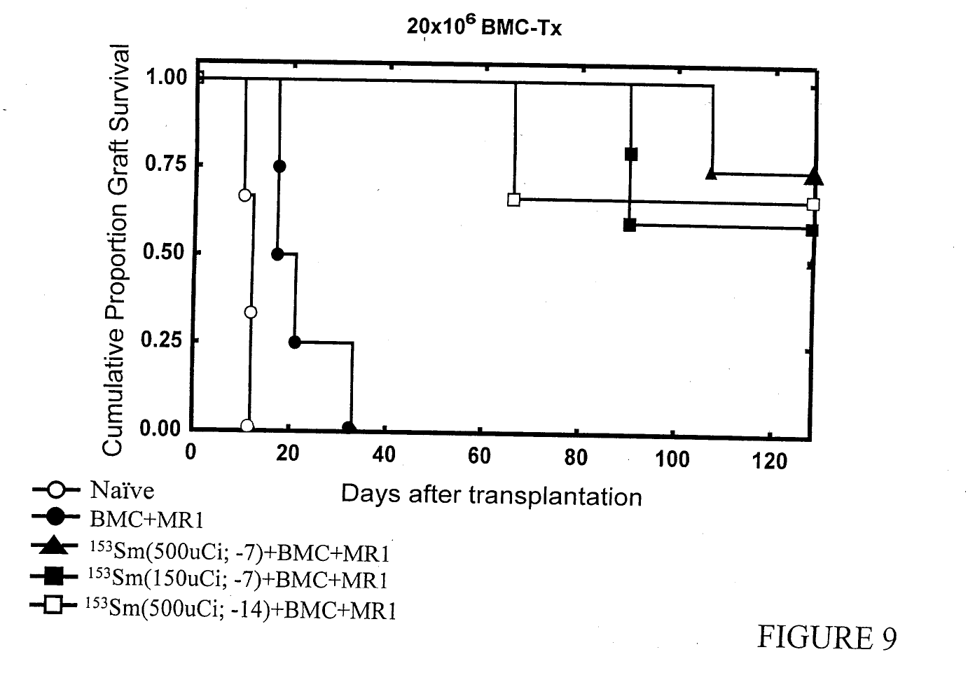

The survival of full thickness tail-derived skin grafts placed on the recipients treated with 20×10⁶ BMC, ¹⁵³Sm-EDTMP, and anti-CD154 mAb, or indicated control groups is shown in FIG. 9. Grafts were prepared 30 days following the last administration of anti-CD154 mAb in the treated animals. Two different donor strain combinations, BALB/c (H-2^{d}), and C3H/J (H-2^{k}) were used. Each recipient received skin grafts from both strains: donor-type, BALB/c (H-2^{d}), as well as third-party, C3H/J (H-2^{k}). Third party grafts were rejected within the same time frame as were donor-specific grafts placed on naïve recipients. Grafts were followed for a minimum of 128 days and were considered rejected when viable tissue was no longer detected at the transplant site. Therefore, tolerance to donor-specific skin grafts is obtained when animals receive a low dose of BMC (20×10⁶), only if ¹⁵³Sm-EDMP is part of the treatment, while co-stimulation alone (along with BMC) is not sufficient to achieve the same result.

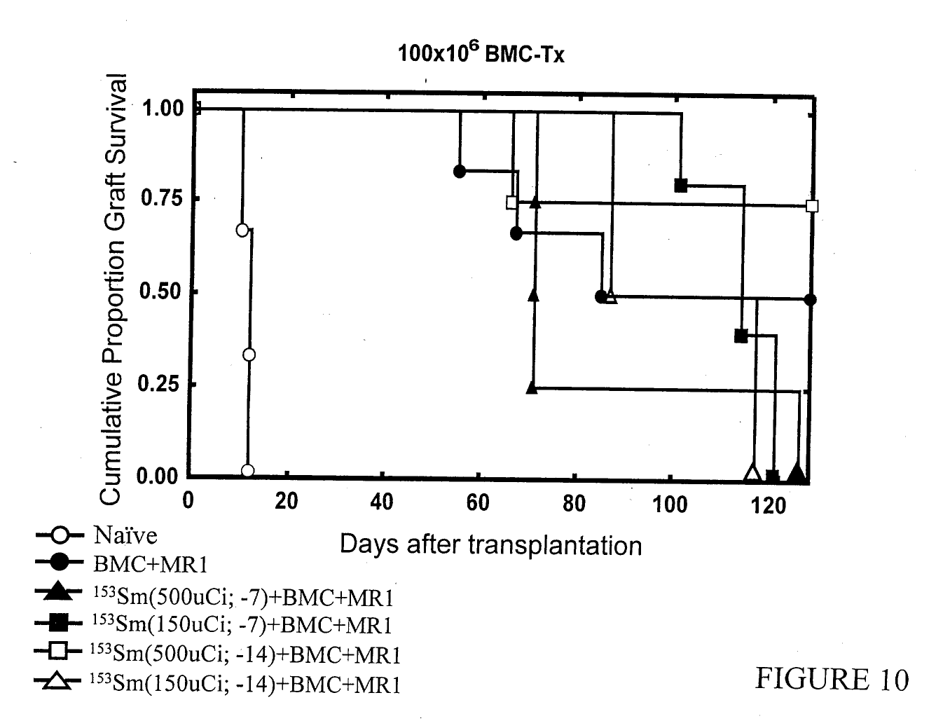

The survival of full thickness tail-derived skin grafts placed on the recipients treated with 100×10⁶ BMC, ¹⁵³ Sm-EDTMP, and anti-CD154 mAb, or indicated control groups is depicted graphically in FIG. 10. Grafts were prepared 30 days following the last administration of anti-CD154 mAb in the treated animals. Two different donor strain combinations, BALB/c (H-2^{d}) and C3H/J (H-2^{k}) were used. Each recipient received skin grafts from both strains: donor-type, BALB/c (H-2^{d}), as well as third-party, C3H/J (H-2^{k}). Third party grafts were rejected within the same time frame as were donor-specific grafts placed on naïve recipients. Grafts were followed for a minimum of 128 days and were considered rejected when viable tissue was no longer detected at the transplant site. Thus, when a high dose of BMC is given (100×10⁶), the enhancing effect of ¹⁵³Sm-EDMP administration is still visible on chimerism levels, that are reproducibly higher, but lost on graft survival since co-stimulatory blockade only (+BMC-Tx) appears similarly efficacious.

The data generated during the instant studies demonstrates one aspect of the invention, wherein high levels of stable long-term chimerism across a full allogeneic barrier can be achieved by a single administration of a bone seeking radioactive compound, such as ¹⁵³Samarium EDTMP, prior to the infusion of allogeneic bone marrow-derived cells. For example, allogeneic bone marrow-derived cells may be infused in the presence of a transient T cell co-stimulatory blockade obtained by administration of anti-CD154 monoclonal antibodies (mAb). A large percent of animals tested, followed for up to 31 weeks post bone marrow transplantation, developed donor-specific tolerance, since these animals kept donor-derived skin grafts for more then 150 days. For a skin graft, maintaining the graft for at least 60 days is preferred, with 100 days being more preferred.

The data indicate that donor-specific hyporesponsiveness can be obtained without harsh cytotoxic pre-conditioning regimens, and therefore, opens extended possibilities for the use of bone marrow transplantation in a clinical setting. Furthermore, the use of bone-seeking radioactive compounds proven effective in enhancing chimerism levels might prove critical in optimizing strategies to achieve hemolymphopoietic chimerism for the treatment of hematological malignancies and disorders, and autoimmune diseases.

While the invention has been illustrated via the preferred embodiments described above, it will be understood that the invention may be practiced employing various modifications evident to those skilled in the art without departing from the spirit and scope of the invention as generally described herein, and as further set forth by the appended claims.

What is claimed is:

1. A method of achieving hemolymphopoietic chimerism comprising:
administering to a recipient a bone seeking radiopharmaceutical;
transplanting bone marrow-derived cells into the recipient; and
transiently suppressing lymphocyte response so as to induce hemolymphopoietic chimerism.

2. The method according to claim 1, wherein the chimerism is induced so as to provide immunological tolerance.

3. The method of claim 2 wherein the immunological tolerance comprises tolerance to at least one member of the group consisting of alloantigens, autoantigens and xenoantigens.

4. The method according to claim 1, wherein the bone seeking radiopharmaceutical is a complex comprising a radionuclide and a ligand.

5. A method of claim 4 wherein the radionuclide is selected from the group consisting of Sm-153, Ho-166, Gd-159, Lu-177, Dy-165, Y-90, In-155m, Re-186, Re-188, Sn-117m, La-140,I-131, Cu-67, Ac-225, Bi-212, Bi-213, At-211, Ra-223, Pm-149, Rh-105; Au-198, Au-199, Dy-166, Sc-47, Yb-175, P-32, Sr-89, Ir-192, Th-149, and Ra-224.

6. A method of claim 4 wherein the ligand is selected from the group consisting of ethylenediaminetetramethylenephosphonic acid (EDTMP), diethylenetriaminepentamethylenephosphonic acid (DTPMP), hydroxyethylethylenediaminetrimethylenephosphonic acid (HEEDTMP), nitrilotrimethylenephosphonic acid (NTMP), tris(2-aminoethyl)aminehexamethylenephosphonic acid (TTHMP), 1-carboxyethylenediaminetetramethylenephosphonic acid (CEDTMP) and bis(aminoethylpiperazine)tetramethylenephosphonic acid (AEPTMP), Ethylenediaminetetraacetic acid (EDTA), 1,4,7,10-tetraazacyclododecane-N,N′,N″,N′″-tetramethylenephosphonic acid (DOTMP), hydroxyethyldiphosphonic acid (HEDP), methylenediphosphonic acid (MDP), diethylenetriaminepentaacetic acid (DTPA), hydroxethylethylenediaminetriacetic acid (HEDTA), and nitrilotriacetic acid (NTA).

7. The method according to claim 4 wherein the complex is selected from the group consisting of Sm-153-EDTMP, Sm-153-DOTMP, Ho-166-EDTMP, Ho-166-DOTMP, Gd-159-EDTMP, Gd-159-DOTMP, Dy-165-EDTMP, Dy-165-DOTMP, Re-186-HEDP, Re-188-HEDP, Sn-117m-DTPA.

8. The method according to claim 1 wherein the method of suppressing lymphocyte response comprises administering at least one biological modifier.

9. The method according to claim 8 wherein the biological modifier is an antibody, a cytokine, an immunosuppressive drug, a peptide, a protein, a nucleic acid or a combination thereof.

10. The method according to claim 8, wherein the biological modifier is at least one antibody that recognizes an antigen selected from the group consisting of CD154, CD4, CD8, CD3, CD5, CD55, CD40, CD40L, B7.1, B7.2, CD28, and LFA-1.

11. A method for decreasing rejection of transplanted organs, tissues or cells comprising:
administering to a recipient a bone seeking radiopharmaceutical;
transplanting bone marrow-derived cells into the recipient;
transiently suppressing lymphocyte response; and
transplanting one or more organs, tissues or cells.

12. The method according to claim 11, wherein the bone seeking radiopharmaceutical is a complex comprising a radionuclide and a ligand.

13. The method of claim 12 wherein the radionuclide is selected from the group consisting of Sm-153, Ho-166, Gd-159, Lu-177, Dy-165, Y-90, In-155m, Re-186, Re-188, Sn-117m, La-140,I-131, Cu-67, Ac-225, Bi-212, Bi-213, At-211, Ra-223, Pm-149, Rh-105; Au-198, Au-199, Dy-166, Sc-47, Yb-175, P-32, Sr-89, Ir-192, Tb-149, and Ra-224.

14. The method of claim 12 wherein the ligand is selected from the group consisting of ethylenediaminetetramethylenephosphonic acid (EDTMP), diethylenetriaminepentamethylenephosphonic acid (DTPMP), hydroxyethylethylenediaminetrimethylenephosphonic acid (HEEDTMP), nitrilotrimethylenephosphonic acid (NTMP), tris(2-aminoethyl)aminehexamethylenephosphonic acid (TTHMP), 1-carboxyethylenediaminetetramethylenephosphonic acid (CEDTMP) and bis(aminoethylpiperazine)tetramethylenephosphonic acid (AEPTMP),. Ethylenediaminetetraacetic acid (EDTA), 1,4,7,10-tetraazacyclododecane-N,N′,N″,N′″-tetramethylenephosphonic acid (DOTMP), hydroxyethyldiphosphonic acid (HEDP), methylenediphosphonic acid (MDP), diethylenetriaminepentaacetic acid (DTPA), hydroxethylethylenediaminetriacetic acid (HEDTA), and nitrilotriacetic acid (NTA).

15. The method according to claim 12 wherein the complex is chosen from the group consisting of Sm-153-EDTMP, Sm-153-DOTMP, Ho-166-EDTMP, Ho-166-DOTMP, Gd-159-EDTMP, Gd-159-DOTMP, Dy-165-EDTMP, Dy-165-DOTMP, Re-186-HEDP, Re-188-HEDP, and Sn-117m-DTPA.

16. The method according to claim 11 wherein the method of suppressing lymphocyte response comprises administering at least one biological modifier.

17. The method according to claim 16 wherein the biological modifier is an antibody, a cytokine, an immunosuppressive drug, a peptide, a protein, a nucleic acid or a combination thereof.

18. The method according to claim 17, wherein the biological modifier is at least one antibody that recognizes an antigen selected from the group consisting of CD154, CD4, CD8, CD3, CD5, CD55, CD40, CD40L, B7.1, B7.2, CD28, and LFA-1.

19. The method according to claim 11 wherein the transplanted organ, tissue or cell comprises liver, heart, lung, kidney, intestine, pancreas, larynx, blood vessels limbs, endocrine organs, skin, islet cells, cornea, nerves, muscles, keratinocytes and keratynocyte precursors, chondrocytes and condrocyte precursors hepatocytes and hepatocyte precursors, myocytes and myoblasts including cardiomyocytes and cardiomyoblasts, neural cells and neural cell precursors, endothelial cells, endocrine cells and endocrine cell precursors, stem cells and cells of different lineage derived from stem cells.

20. A method for treating diabetes comprising the method of claim 19, wherein the transplanted cells are islet cells.

21. A method to treat autoimmune disease comprising:
administering to a recipient a bone seeking radiopharmaceutical;
transplanting bone marrow-derived cells into the recipient; and
transiently suppressing lymphocyte response.

22. The method of claim 21 wherein the transplanted bone marrow-derived is autologous and is either unmanipulated or is depleted of mature T-lymphocytes prior to transplantation.

23. The method according to claim 21, wherein the bone seeking radiopharmaceutical is a complex comprising a radionuclide and a ligand.

24. The method of claim 23 wherein the radionuclide is selected from the group consisting of Sm-153, Ho-166, Gd-159, Lu-177, Dy-165, Y-90, In-155m, Re-186, Re-188, Sn-117m, La-140,I-131, Cu-67, Ac-225, Bi-212, Bi-213, At-211, Ra-223, Pm-149, Rh-105; Au-198, Au-199, Dy-166, Sc-47, Yb-175, P-32, Sr-89, Ir-192, Tb-149, and Ra-224.

25. The method of claim 23 wherein the ligand is selected from the group consisting of ethylenediaminetetramethylenephosphonic acid (EDTMP), diethylenetriaminepentamethylenephosphonic acid (DTPMP), hydroxyethylethylenediaminetrimethylenephosphonic acid (HEEDTMP), nitrilotrimethylenephosphonic acid (NTMP), tris(2-aminoethyl)aminehexamethylenephosphonic acid (TTHMP), 1-carboxyethylenediaminetetramethylenephosphonic acid (CEDTMP) and bis(aminoethylpiperazine)tetramethylenephosphonic acid (AEPTMP),. Ethylenediaminetetraacetic acid (EDTA), 1,4,7,10-tetraazacyclododecane-N,N′,N″,N′″-tetramethylenephosphonic acid (DOTMP), hydroxyethyldiphosphonic acid (HEDP), methylenediphosphonic acid (MDP), diethylenetriaminepentaacetic acid (DTPA), hydroxethylethylenediaminetriacetic acid (HEDTA), and nitrilotriacetic acid (NTA).

26. The method according to claim 23 wherein the complex is chosen from the group consisting of Sm-153-EDTMP, Sm-153-DOTMP, Ho-166-EDTMP, Ho-166-DOTMP, Gd-159-EDTMP, Gd-159-DOTMP, Dy-165-EDTMP, Dy-165-DOTMP, Re-186-HEDP, Re-188-HEDP, and Sn-117m-DTPA.

27. The method according to claim 21 wherein the method of suppressing lymphocyte response comprises administering at least one biological modifier.

28. The method according to claim 27 wherein the biological modifier is an antibody, a cytokine, an immunosuppressive drug, a peptide, a protein, a nucleic acid or a combination thereof.

29. The method according to claim 28, wherein the biological modifier is at least one antibody that recognizes an antigen selected from the group consisting of CD154, CD4, CD8, CD3, CD5, CD55, CD40, CD40L, B7.1, B7.2, CD28, and LFA-1.

30. The method according to claim 21 wherein the autoimmune disease is selected from diseases of the nervous system, the eye, cardiac system, respiratory system, urogenital system, gastrointestinal system, blood, blood vessels, endocrine glands, skin, and musculoskeletal system.

31. The method according to claim 30 wherein the autoimmune disease is selected from rheumatoid arthritis, ankylosing spondilytis polymyositis, dermatomyositis systemic lupus erythematosus, vasculitides, Goodpasture's syndrome, Wegener granulomatosis uveitis, Sjogren's syndrome, Bechet's disease, autoimmune myocarditis and perycarditis, multiple sclerosis, inflammatory bowel disease, Crohn's disease, ulcerative colitis, autoimmune gastritis, autoimmune hepatitis primary biliary chirrosis, diabetes, autoimmune thyroid disease, Graves disease, Hashimoto thyroiditis, Addison's disease, ipoparathyroidism, autoimmune hypophysitis, ovaritis, myastenia gravis, alopecia areata universalis, vitiligo, psoriasispemphigus p, p scleroderma, and autoimmune diseases of the blood.
